(19) United States
(12) Patent Application Publication
(10) Pub. No.: US 2011/0053927 A1
(43) Pub. Date: Mar. 3, 2011

(54) NANOPARTICLE FORMATION VIA RAPID PRECIPITATION

(75) Inventors: Hsien-Hsin Tung, Edison, NJ (US); Lei Wang, Ambler, PA (US); Santipharp Panmai, North Wales, PA (US); Michael Riebe, Collegeville, PA (US)

(73) Assignee: Merck Sharp & Dohme Corp., Rahway, NJ (US)

(21) Appl. No.: 12/989,148

(22) PCT Filed: Apr. 20, 2009
(86) PCT No.: PCT/US09/41083

§ 371 Date: Oct. 22, 2010

Related U.S. Application Data
(60) Provisional application No. 61/125,211, filed on Apr. 23, 2008.

(51) Int. Cl. A61K 31/5377; A61P 1/08; A61K 9/14; B01J 19/10; B82Y 40/00

(52) U.S. Cl. 514/230.8; 204/157.62; 977/895

(57) ABSTRACT

The invention encompasses a method for making nano-sized particles of water-insoluble pharmaceuticals comprising: (1) dissolving the water-insoluble pharmaceutical in a water-miscible solvent, optionally with water and inactive pharmaceutical ingredients, to make a solution; (2) rapidly mixing the solution with an anti-solvent which creates a high level of supersaturation, wherein the anti-solvent is water with optional inactive pharmaceutical ingredients; (3) simultaneously applying energy to the resulting mixture during the mixing of solution and anti-solvent as nano-sized drug particles precipitate out and form a slurry mixture under supersaturation; and (4) optionally isolating the nano-sized particles of water-insoluble pharmaceuticals from the slurry mixture.

### BACKGROUND OF THE INVENTION

Water insoluble drugs, also called lipophilic, hydrophobic, etc., constitute a growing segment of the discovery and development portfolio of pharmaceutical industries. Since the first step in the oral absorption of drug is its dissolution in the gastrointestinal lumen contents, poor aqueous solubility is rapidly becoming the leading hurdle for formulation scientists working on oral delivery of drug compounds.

To improve the dissolution rate of water insoluble drugs, one proven technique is to reduce particle size down to submicron domain (F. Kesisoglou, etc., Advanced Drug Delivery Reviews, 2007, 59, p 631-644). In general, formation of submicron or nanoparticles can be done through top-down approach in which particles are milled to the desired size range, or bottom-up approach in which small nuclei are generated under high supersaturation and grown to the desired size range. Both approaches have their advantages and limitations. For example, milling generally is not sensitive to the physical properties of drugs, but milling time cycle can be lengthy which leads to heavy capital investment. Precipitation is typically highly productive, but conditions for formation of nanoparticles are highly dependent upon the physical properties of drugs.

For bottom-up approach, the typical method is described as flash nanoprecipitation (Aust. J. Chem. 2003, 56, p 1021; US 2004/0091546 A1) or mixing-T precipitation (Angew. Chem. Int. Ed. 2001, 40, p 4330). In this approach, one stream of batch solution and one stream of anti-solvent are impinged upon each other at high velocities within the impinging jet or mixing-T device. A high local turbulence is generated upon impingement of these two streams and it creates a clear uniform highly unstable supersaturated solution mixture in milliseconds. Solid drug particles or oily drug droplets will rapidly form from this unstable highly supersaturated solution, and supersaturation is released accordingly. Depending upon drug physical properties and operating conditions, the solid drug particles or oily drug droplets may further transform into a more stable solid drug particles upon aging.

In essence, the particle formation process consists of two key stages with an optional third aging stage. The first stage is the formation of a clear uniform highly unstable supersaturated solution. The second stage is the formation of solid drug particles or oily drug droplets under high supersaturation. The optional third aging stage is the transformation of drug particles/droplets to stable drug particles upon aging. As described in the impinging jet or mixing-T approach, energy is only applied during the first stage where streams are impinged upon each other. No energy is applied during the second stage where particles are being formed, or the third stage.

A similar precipitation process scheme was disclosed in WO 03/032951 A1 which includes a recycle loop for continuous operation. Within the loop, the clear batch solution is injected into the recycle loop and mixed rapidly with the recycling stream. Rapid mixing at the point of addition can be achieved via some type of mixing device, for example a mixing-T, or a high speed rotor-stator homogenizer, etc. The key requirement of mixing at the point of addition is that the batch solution and the recycle stream should be mixed so rapidly that a uniform highly supersaturated solution can be generated among the existing particles in the recycle slurry before the formation of new particles. As described in this recycle loop approach, energy is applied only at the first stage where high supersaturation is generated. No energy is applied during the second stage of formation of new particles under high supersaturation or third aging stage.

An hybrid approach combining both bottom-up and top-down approaches for the formation of nanoparticles was disclosed in U.S. Pat. No. 6,607,784 (2003). Similar to two cases described above, at the first stage energy is applied at the first stage to generate high supersaturation. At the second stage, particles or droplets are generated under high supersaturation without energy. However, at the third aging stage, energy is applied to break the particles. Energy also facilitates the transformation of solid particles or oily droplets to more these particles/droplets at the third stage.

The current invention encompasses an energy-efficient true bottom-up method for the generation of nano-sized particles. In contrast to previous examples, in this invention energy is applied during the second stage where drug particles are fowled under supersaturation. The underlying mechanism of the current invention is considered to be secondary nucleation (A. Mersmann, Crystallization Technology Handbook, 2001, Chapters 5, Secondary Nucleation). That is, in the presence of supersaturation, energy is applied to enhance the secondary nucleation rate, thus the formation of nanoparticles. This is a bottom-up approach. The nuclei are generated and grow under supersaturation.

Fundamentally, this is very different from previous approaches. In the earlier cases presented, if at the second stage particles were formed from a clear highly supersaturated solution, the key mechanism is primary nucleation during this stage, rather than secondary nucleation. If there is a recycle loop, at the second stage particles are formed under high supersaturation in the presence of particles, the key mechanism becomes secondary nucleation. However, no energy was applied to enhance the nucleation rate. If an optional energy was applied at the third stage, the key mechanism is actually particle breakage. This is a top-down rather than bottom-up approach. This is clearly different from the current invention.

### SUMMARY OF THE INVENTION

The invention encompasses a method for making nano-sized particles of water-insoluble pharmaceuticals comprising: (1) dissolving the water-insoluble pharmaceutical in a water-miscible solvent, optionally with water and inactive pharmaceutical ingredients, to make a solution; (2) rapidly mixing the solution with an anti-solvent which creates a high level of supersaturation, wherein the anti-solvent is water with optional inactive pharmaceutical ingredients; (3) simultaneously applying energy to the resulting mixture during the mixing of solution and anti-solvent as nano-sized drug particles precipitate out and form a slurry mixture under supersaturation; and (4) optionally isolating the nano-sized particles of water-insoluble pharmaceuticals from the slurry mixture.

### BRIEF DESCRIPTION OF THE DRAWINGS

FIG. 1—Set-up for impinging jet crystallization.

FIG. 2—Mean Particle Size of Examples 1, 2, and 3.

FIG. 3—Impinging jet crystallization with recycle loop.

FIG. 4—Impinging jet crystallization with recycle loop and sonication horn.

FIG. 5—Particle Size Distribution of Examples 1, 2, and 3 after sonication.

### DETAILED DESCRIPTION OF THE INVENTION

The invention encompasses a method for making nano-sized particles of water-insoluble pharmaceuticals comprising: (1) dissolving the water-insoluble pharmaceutical in a water-miscible solvent, optionally with water and inactive pharmaceutical ingredients, to make a solution; (2) rapidly mixing the solution with an anti-solvent which creates a high level of supersaturation, wherein the anti-solvent is water with optional inactive pharmaceutical ingredients; (3) simultaneously applying energy to the resulting mixture during the mixing of solution and anti-solvent as nano-sized drug particles precipitate out and form a slurry mixture under supersaturation; and (4) optionally isolating the nano-sized particles of water-insoluble pharmaceuticals from the slurry mixture.

In an embodiment, the invention encompasses the above method wherein the water-miscible solvent is selected from the group consisting of: alcohols, ketones, acetonitrile, and tetrahydrofuran. An alcohol means for example methanol and ethanol. A ketone means for example acetone.

In another embodiment, the invention encompasses the above method wherein the inactive pharmaceutical ingredients are selected from the group consisting of: ionic surfactants, non-ionic surfactants, hydrophobic polymers, hydrophilic polymers and amphiliphilic polymers.

In another embodiment, the invention encompasses the above method wherein the solution is rapidly mixed using a device selected from the group consisting of: a jet impinging device, a mixing-T, a vortex mixer and a high speed rotor/stator homogenizer.

In another embodiment, the invention encompasses the above method wherein energy is applied using a device selected from the group consisting of: ultrasound homogenizer, high pressure homogenizer and high speed rotor/stator homogenizer.

In another embodiment, the invention encompasses the above method wherein the nano-sized particles of water-insoluble pharmaceutical are isolated by a technique selected from the group consisting of lyophilization, ultracentrifugation, membrane filtration and spray coating on inactive pharmaceutical ingredients.

The term “nano-sized particles” means the mean size of particles is less than 1.0 um.

The term “water-insoluble drug or pharmaceutical” means a pharmaceutical active ingredient that is insoluble or nearly insoluble in water with a dose number greater than 10. The dose number is defined as follows:

Dose number=(theoretical dose in mg/250 ml)/water solubility

For example, if the theoretical dose of the drug is 25 mg per dose. If its water solubility is 0.01 mg/ml, the dose number would be (25/250)/0.01=10. Examples of water insoluble pharmaceuticals include lovastatin (water solubility<0.01 mg/ml of water) and simvastatin (water solubility<0.01 mg/ml of water). At a hyphothetic dose of 25 mg/dose, both lovastatin and simvastatin will have a dose number great than 10. Another example of a water in-soluble pharmaceutical is the compound of Formula I (MK-0869):

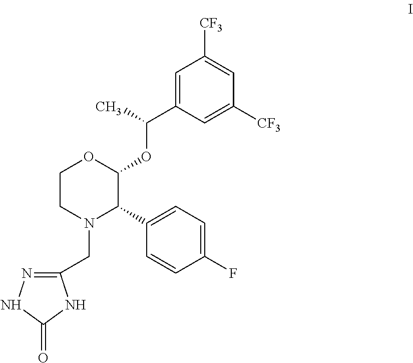

MK-869 is also known by the generic name aprepitant and is commercially available and sold under the trade name EMEND® (Merck & Co., Inc.).

The term “solubility” means the amount of drug dissolved per unit volume of solvent or solvent mixture at equilibrium.

The term “supersaturation” means the solution concentration exceeds the equilibrium solubility of drug. Supersaturation can be generated by mixing a solvent containing dissolved drug and anti-solvent which has a low solubility of the drug, and is well known for those having ordinary skills in the art.

The term “water-miscible solvent” means solvent which is miscible with water at a solvent composition less than 50 wt % of the solvent/water mixture. Examples of water miscible solvents include alcohols such as methanol, ethanol; ketones such as acetone and various other solvents such as acetonitrile, and tetrahydrofuran (THF) and the like.

The term “inactive pharmaceutical ingredients” means excipients which are accepted by FDA for pharmaceutical formulation of drugs. Examples of inactive pharmaceutical ingredient include surfactants such as sodium lauryl sulfate, poloxamer, HPC, HPMC, HPMCAS etc.

The term “rapidly mixing” can be accomplished using a variety of devices such as a jet impinging device, a mixing-T, a vortex mixer, or a high speed rotor/stator homogenizer, etc.

The devices and methods for operating these devices are well known for those having ordinary skills in the art. An impinging jet device, for example, is described in U.S. Pat. No. 5,314,506.

The term “energy” can be accomplished using a variety of device such as impinging jet, ultrasound homogenizer, high pressure homogenizer, or high speed rotor/stator homogenizer, etc. The devices apply intensive energy, and result in rapid and vigorous mixing. Methods for operating these devices are well known for those having ordinary skills in the art. In contrast, overhead agitator, magnetic bar, static mixer, or recycling stream are much less energy intensive and is not classified as “energy” device in this invention.

The nano-sized particles of water-insoluble pharmaceutical can be isolated by a variety of techniques, such as lyophilization, ultracentrifugation, membrane filtration, or spray coating on inactive pharmaceutical ingredients. The methods are well known for those having ordinary skills in the art.

The invention will now be illustrated by the following non-limiting examples:

### EXAMPLE 1

### Impinging Jet Crystallization with Optional Sonication after Crystallization

600 mgs of compound of Formula I solid, along with 120 mgs of hydroxylpropyl cellulose polymer of grade HPC-SL, and 6 mgs of sodium lauryl sulfate, were dissolved in 4 grams of acetone and 1 grams of water in a glass vial at room temperature under magnetic bar stirring. All solids were dissolved in 20 minutes.

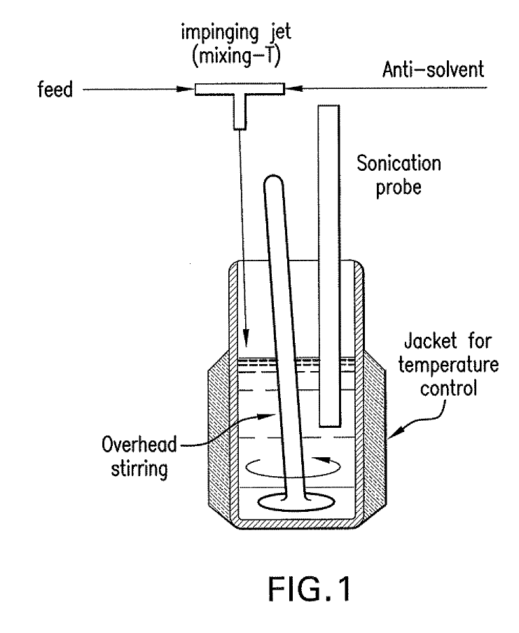

After the complete dissolution, the clear solution was mixed with 70 ml of water (as anti-solvent) through an impinging jet device, i.e. mixing-T, over 10 minutes as shown in FIG. 1. The orifice size of the mixing-T is 150 μm for all three ports. The hold-up of the mixing-T is approximate 20 nanoliter. The linear velocity for the clear solution of dissolved drug through the mixing-T orifice is calculated to be approximate 0.5 m/s. The linear velocity for the antisolvent through the mixing-T orifice is calculated to be approximate 7 m/s. The calculated residence time for mixing two streams in the mixing-T is estimated to be less than 1 millisecond.

The highly supersaturated mixture is transferred through a 1/16″ OD, 250 μm ID tubing from the mixing-T to a jacketed 50 ml crystallizer with an overhead agitator for stirring. The crystallizer was maintained at 3-5° C. During the experiment, solids precipitate out rapidly upon exiting the mixing-T device.

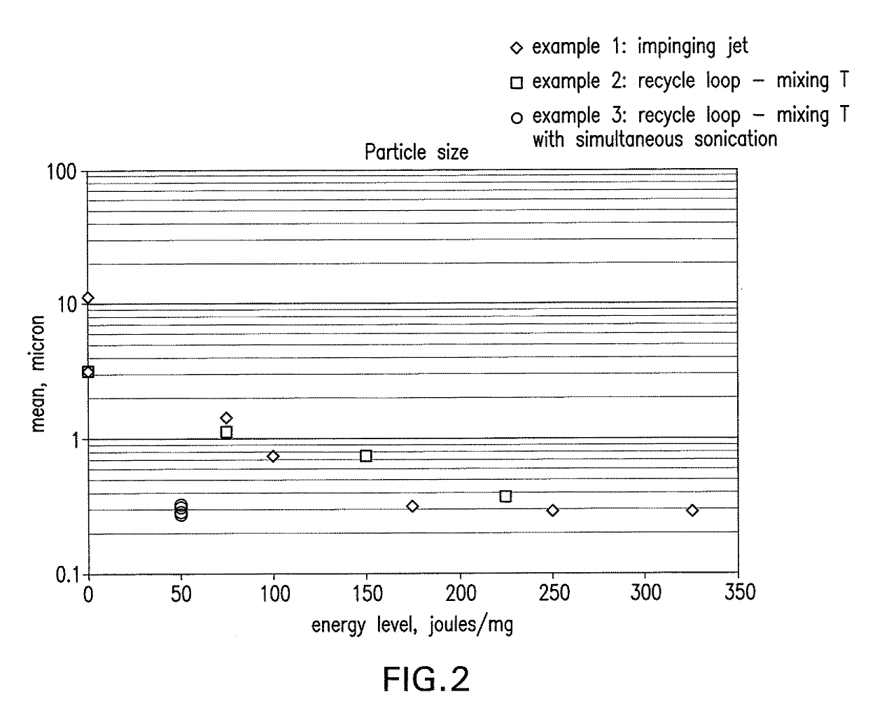

After completing the transfer, slurry in the crystallizer was sonicated using Branson sonifier model 250. The temperature was maintained at 5-10° C. during the sonication. Samples were taken over time for particle size measurement using Horiba LA-910 Laser Scattering Particle Size Distribution Analyzer. Results are shown in FIG. 2.

As shown in FIG. 2, impinging jet crystallization alone did not generate nanoparticles. Applying energy during the third stage aging period was able to reduce the particle to nanosize range.

### EXAMPLE 2

### Impinging Jet Crystallization with Recycle Loop and Optional Sonication after Crystallization

Similar to example 1, 600 mgs of compound of Formula I solid, along with 120 mgs of hydroxyl propyl cellulose polymer of grade HPC-SL, and 6 mgs of sodium lauryl sulfate, were dissolved in 4 grams of acetone and 1 grams of water in a glass vial at room temperature. All solids were dissolved after stirring for 20 minutes.

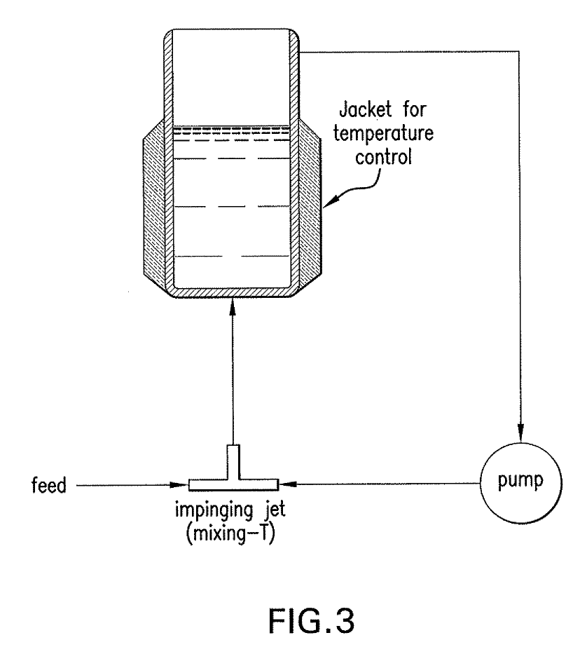

The solution containing dissolved compound was added into a recirculation loop with a mixing-T device as shown in FIG. 3. The clear batch solution was added through the mixing-T over 10 minutes. The diameter of the addition port is 120 um and the calculated linear velocity of batch solution in addition port is approximate 0.6 m/s. The antisolvent of 70 ml was circulated through the loop. The recycle stream temperature was maintained at 3-5° C. The recycle stream was circulated at a flow rate of 1600 ml/min through the mixing T. The diameter of two other ports of mixing-T in the loop was ⅛″. The calculated linear velocity is approximately 4 m/s through the mixing-T. These conditions ensured that a similar intensive mixing pattern within the mixing-T was maintained as in example 1.

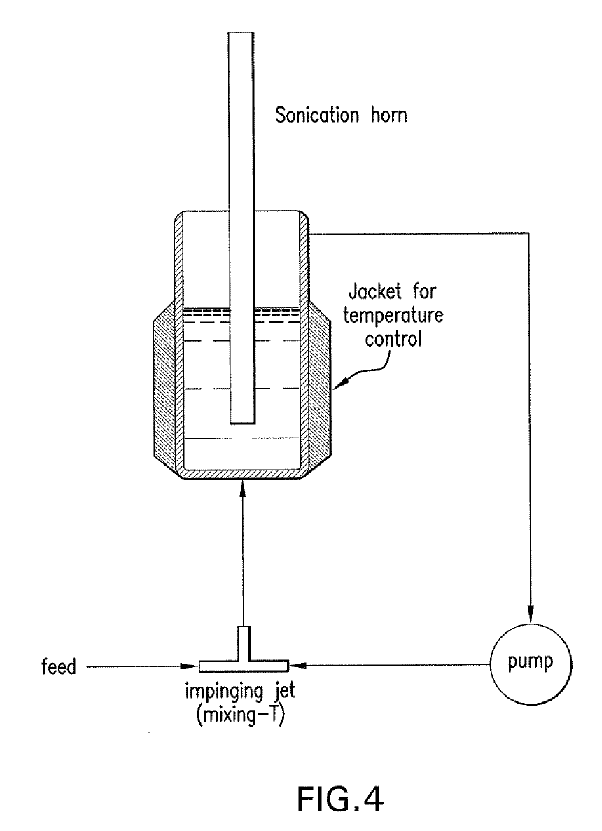

During the addition, the solution in the loop turned from clearness to cloudiness gradually. After completing the addition, sonication horn was placed into the recycle loop as shown in FIG. 4. The slurry mixture in the crystallizer was sonicated using Branson digital sonifier model 250 over time while maintaining the batch temperature around 5° C. Samples were taken periodically for particle size measurement using Horiba LA-910 Laser Scattering Particle Size Distribution Analyzer. Particle size profile over time is shown in FIG. 2.

As shown in FIG. 2, particle size of recycle loop material is similar to that from example 1. Applying energy during the third stage aging period reduces particle size.

### EXAMPLE 3

### Impinging Jet Crystallization with Recycle Loop and Simultaneous Sonication during Crystallization

Similar to example 2, a sonication probe was installed in the recycle loop as shown in FIG. 4. All experimental conditions are identical to example 2 with the exception that sonication was applied simultaneously during the addition of batch solution. After completing the addition, a slurry sample was taken for particle size analysis using Horiba LA-910 Laser Scattering Particle Size Distribution Analyzer. Particle size is shown in FIG. 2.

As shown in FIG. 2, simultaneous sonication during the impinging crystallization generated similar or smaller size of nanoparticles. However, significant less amount of energy is required.

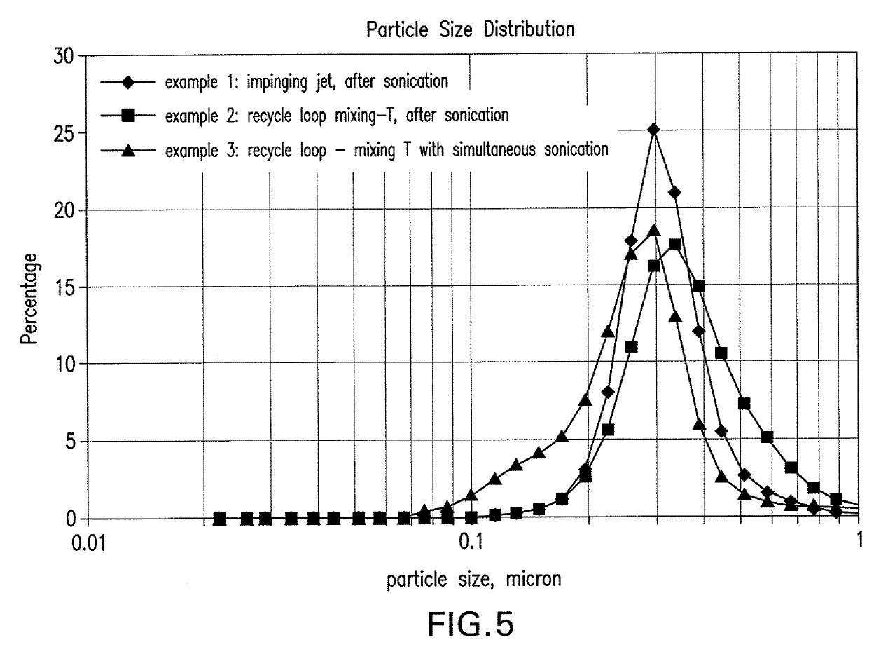

To further demonstrate the difference among these approaches, FIG. 5 plots the final particle size distribution after sonication example 1 after 320 joules/mg of sonication, example 2 after 220 joules/mg of sonication and example 3 after 50 joules/mgl of sonication. As shown in FIG. 5, sample from example 3 possess significantly more fine particles than samples of example 1 and 2. This further reinforces the claimed advantage of the current invention over prior approaches that applying energy to enhance secondary nucleation under supersaturation is more effective to generate nanoparticles than applying energy to break particles afterward.

What is claimed is:

1. A method for making nano-sized particles of water-insoluble pharmaceuticals comprising: (1) dissolving the water-insoluble pharmaceutical in a water-miscible solvent, optionally with water and inactive pharmaceutical ingredients, to make a solution; (2) rapidly mixing the solution with an anti-solvent which creates a high level of supersaturation, wherein the anti-solvent is water with optional inactive pharmaceutical ingredients; (3) simultaneously applying energy to the resulting mixture during the mixing of solution and anti-solvent as nano-sized drug particles precipitate out and form a slurry mixture under supersaturation; and (4) optionally isolating the nano-sized particles of water-insoluble pharmaceuticals from the slurry mixture.

2. The method according to claim 1 wherein the water-miscible solvent is selected from the group consisting of alcohols, ketones, acetonitrile, and tetrahydrofuran.

3. The method according to claim 1 wherein the inactive pharmaceutical ingredients are selected from the group consisting of: ionic surfactants, non-ionic surfactants, hydrophobic polymers, hydrophilic polymers and amphiliphilic polymers.

4. The method according to claim 1 wherein the solution is rapidly mixed using a device selected from the group consisting of a jet impinging device, a mixing-T, a vortex mixer and a high speed rotor/stator homogenizer.

5. The method according to claim 1 wherein energy is applied using a device selected from the group consisting of: ultrasound homogenizer, high pressure homogenizer and high speed rotor/stator homogenizer.

6. The method according to claim 1 wherein the nano-sized particles of water-insoluble pharmaceutical are isolated by a technique selected from the group consisting of lyophilization, ultracentrifugation, membrane filtration and spray coating on inactive pharmaceutical ingredients.
